(19) United States
(12) Patent Application Publication
(10) Pub. No.: US 2006/0004289 A1
(43) Pub. Date: Jan. 5, 2006

(54) High sensitivity capacitive micromachined ultrasound transducer

(76) Inventors: Wei-Cheng Tian, Clifton Park, NY (US); Warren Lee, Clifton Park, NY (US); Lowell Scott Smith, Niskayuna, NY (US); Ye-Ming Li, Schenectady, NY (US); Jie Sun, Schenectady, NY (US)

Correspondence Address: Patrick S. Yoder;FLETCHER YODER, P.O. Box 692289, Houston, TX, 77269-2289, US

(21) Appl. No.: 10/881,924

(22) Filed: Jun. 30, 2004

(51) Int. Cl. A61B 8/14

(52) U.S. Cl. 600/459

(57) ABSTRACT

A capacitive micromachined ultrasound transducer (cMUT) comprises a lower electrode. Furthermore, the cMUT includes a diaphragm disposed adjacent to the lower electrode such that a gap having a first gap width is formed between the diaphragm and the lower electrode. Additionally, the cMUT includes at least one element formed in the gap, where the at least one element is arranged to provide a second gap width between the diaphragm and the lower electrode.

### BACKGROUND

The invention relates generally to medical imaging systems, and more specifically to capacitive micromachined ultrasound transducers (cMUTs).

Transducers are devices that transform input signals of one form into output signals of a different form. Commonly used transducers include light sensors, heat sensors, and acoustic sensors. An example of an acoustic sensor is an ultrasonic transducer, which may be implemented in medical imaging, non-destructive evaluation, and other applications.

Currently, one form of an ultrasonic transducer is a capacitive micromachined ultrasound transducer (cMUT). A cMUT cell generally includes a substrate that contains a lower electrode, a diaphragm suspended over the substrate by means of support posts, and a metallization layer that serves as an upper electrode. The lower electrode, diaphragm, and the upper electrode define a cavity. In conventional cMUT devices, the gap between the upper and lower electrodes of the cMUT cell is designed to be uniform and narrow in order to increase the sensitivity when the cMUT transceiver is employed as a receiver. However, the small cavity depth limits the maximum amplitude of the diaphragm displacement when the cMUT transceiver is used as a transmitter. Therefore, in order to increase the amplitude of the transmitted pulse, it may be desirable for the transmitting cMUT to have a larger gap between the upper and lower electrodes to allow a larger diaphragm deflection.

Further, it may be desirable to enhance the sensitivity and performance of the cMUT during operation as a transmitter and a receiver. Also, it may be desirable to actively control the acoustic area (gap) and cavity depth of the cMUT.

### BRIEF DESCRIPTION

Briefly, in accordance with one embodiment of the present technique a capacitive micromachined ultrasound transducer (cMUT) cell is presented. The cMUT includes a lower electrode. Furthermore, the cMUT includes a diaphragm disposed adjacent to the lower electrode such that a gap having a first gap width is formed between the diaphragm and the lower electrode. Additionally, the cMUT includes at least one element formed in the gap, where the at least one element is arranged to provide a second gap width between the diaphragm and the lower electrode.

In accordance with another embodiment of the present technique, a cMUT cell is presented. The cMUT includes a lower electrode comprising a topside and a bottom side. In addition, a plurality of support posts is disposed on the topside of the lower electrode and configured to define a cavity. Furthermore, a diaphragm is disposed on the plurality of support posts to provide a gap bounded by the diaphragm and the lower electrode. Additionally, the cMUT includes an upper electrode disposed on the topside of the diaphragm. In addition, the cMUT includes at least one element formed in the cavity and configured to provide a gap width between the lower electrode and the upper electrode, which is less than the depth of the cavity.

In accordance with another aspect of the present technique, a method for fabricating a cMUT is presented. The method includes forming a plurality of support posts on a lower electrode to define a cavity between the support posts. Additionally, the method includes forming at least one element in the cavity. In addition, the method includes disposing a diaphragm on the plurality of support posts to form a gap between the lower electrode and the diaphragm. Moreover, the method includes disposing an upper electrode on the diaphragm.

In accordance with an aspect of the present technique a cMUT cell structure is presented. The cMUT cell structure includes a first cell configured to operate in a receive mode, where the first cell comprises a lower electrode and an upper electrode. Furthermore, the cMUT cell structure includes a second cell configured to operate in a transmit mode, where the second cell comprises a lower electrode and an upper electrode. Additionally, the cMUT cell structure includes a plurality of support posts arranged to form cavities therebetween in each of the first cell and the second cell. The cMUT cell structure further comprises a plurality of diaphragms disposed on the support posts. In addition, the cMUT cell structure includes at least one of a protruding element and a receding element formed in a cavity of the first cell and the second cell.

In accordance with a further aspect of the present technique, a method for fabricating a cMUT cell structure is presented. The method includes fabricating a first cell configured to operate in a receive mode, where the first cell includes a lower electrode and an upper electrode. Additionally, the method includes fabricating a second cell configured to operate in a transmit mode, where the second cell includes a lower electrode and an upper electrode.

In accordance with an aspect of the present technique, a system including a cMUT and a resistor coupled to the cMUT is presented. Furthermore, the system includes a bias voltage bank, where the bias voltage bank is coupled to the resistor. In addition, the system includes a multiplexer, where the multiplexer is coupled to the resistor. Additionally, the system includes a switch coupled to the multiplexer, where the switch is configured to control modes of operation of the cMUT. The system also includes control circuitry coupled to the switch, where the control circuitry is configured to control operation of the bias voltage bank and the switch. Furthermore, the system includes a pulser coupled to the switch, where the pulser is configured to generate alternating current excitation pulses. Also, the system includes a low noise amplifier coupled to the switch, where the low noise amplifier is configured to enhance signals.

### DRAWINGS

These and other features, aspects, and advantages of the present invention will become better understood when the following detailed description is read with reference to the accompanying drawings in which like characters represent like parts throughout the drawings, wherein:

FIG. 1 is a cross-sectional side view illustrating an exemplary embodiment of a cMUT transceiver comprising a ring stud and operating in a transmit mode according to aspects of the present technique;

FIG. 2 is a cross-sectional side view illustrating an exemplary embodiment of the cMUT transceiver of FIG. 1 comprising a ring stud and operating in a receive mode according to aspects of the present technique;

FIG. 3 is a cross-sectional top view of the cMUT transceiver of FIG. 1 along cross-sectional line 3-3;

FIG. 4 is a cross-sectional side view of an alternate exemplary embodiment of the cMUT transceiver of FIG. 1 comprising a single stud according to aspects of the present technique;

FIG. 5 is a cross-sectional side view of another exemplary embodiment of the cMUT transceiver of FIG. 1 comprising an array of studs according to aspects of the present technique;

FIG. 6 is a cross-sectional side view of another exemplary embodiment of the cMUT transceiver of FIG. 1 comprising a well according to aspects of the present technique;

FIG. 7 is a cross-sectional side view of an alternate exemplary embodiment of the cMUT transceiver of FIG. 2 comprising a single stud according to aspects of the present technique;

FIG. 8 is a cross-sectional side view of another exemplary embodiment of the cMUT transceiver of FIG. 2 comprising an array of studs according to aspects of the present technique;

FIG. 9 is a cross-sectional side view of another exemplary embodiment of the cMUT transceiver of FIG. 2 comprising a well according to aspects of the present technique;

FIG. 10 is a cross-sectional top view of the cMUT transceiver of FIG. 4 along cross-sectional line 10-10;

FIG. 11 is a cross-sectional top view of the cMUT transceiver of FIG. 5 along cross-sectional line 11-11;

FIG. 12 is a cross-sectional top view of the cMUT transceiver of FIG. 6 along cross-sectional line 12-12;

FIG. 13 is a cross-sectional side view illustrating an exemplary embodiment of a dual cavity cMUT unit cell according to aspects of the present technique;

FIG. 14 is a cross-sectional side view illustrating an exemplary embodiment of an alternate configuration of the dual cavity cMUT unit cell of FIG. 13 according to aspects of the present technique;

FIGS. 15-20 illustrate an exemplary process of fabricating the cMUT cell of FIG.1;

FIGS. 21-26 illustrate an alternate exemplary process of fabricating the cMUT cell of FIG. 1;

FIGS. 27-32 illustrate another exemplary process of fabricating the cMUT cell of FIG.1;and

FIG. 33 is a block diagram of a system implementing cMUT transceivers according to one aspect of the present technique.

### DETAILED DESCRIPTION

In many fields, such as medical imaging and non-destructive evaluation, it may be desirable to utilize ultrasound transducers that enable the generation of high quality diagnostic images. High quality diagnostic images may be achieved by means of ultrasound transducers, such as, capacitive micromachined ultrasound transducers, that exhibit high sensitivity to low level acoustic signals at ultrasonic frequencies. The techniques discussed herein address some or all of these issues.

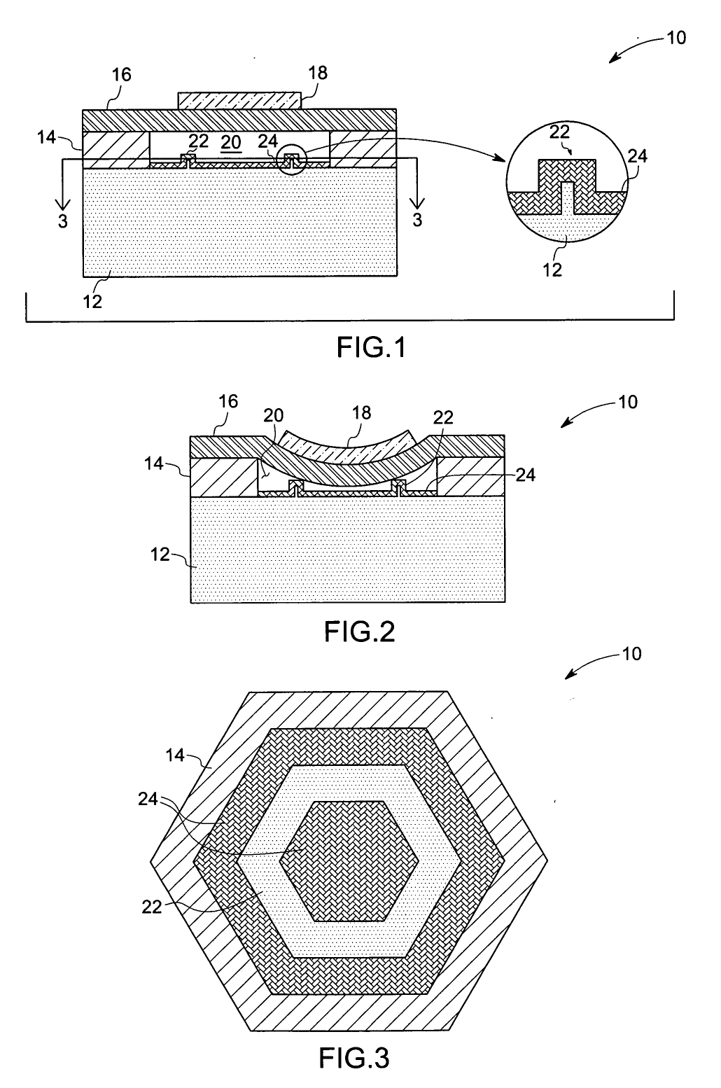

Turning now to FIG. 1, a side view of a cross-section of an embodiment of a capacitive micromachined ultrasound transducer (cMUT) transceiver 10 is illustrated. As will appreciated by one skilled in the art, the figures are for illustrative purposes and are not drawn to scale. FIG. 1 depicts the cMUT transceiver 10 operating in a transmit mode. The cMUT transceiver 10 comprises a lower electrode 12, having a topside and a bottom side, which may be disposed on a substrate (not shown). The thickness of the lower electrode 12 may be, for example, approximately in the range of 20 to 500 micrometers. A plurality of support posts 14, comprising a topside and a bottom side, may be disposed on the topside of the lower electrode 12. Alternatively, the plurality of support posts 14 may be disposed directly on the substrate. The support posts 14 may be configured to define a cavity 20. Generally, the height of the support posts 14 is on the order of tenths to few micrometers (μm). Also, the support posts 14 may be made of material, such as, but not limited to, silicon oxide or silicon nitride. Additionally, a membrane or diaphragm 16 may be disposed on the topside of the plurality of support posts 14. In addition, depending on the micromachining methods employed to fabricate the cMUT, the diaphragm 16 may be fabricated employing materials such as, but not limited to, silicon nitride, silicon oxide, single crystal silicon, epitaxy silicon, polycrystalline silicon, and other semiconductor materials. The thickness of the diaphragm 16 may be, for example, approximately in the range of 0.1 to 5 micrometers. The cMUT transceiver 10 may include an upper electrode 18 comprising a topside and a bottom side, where the upper electrode 18 may be disposed on the topside of the diaphragm 16. The thickness of the upper electrode 18 may be, for example, approximately in the range of 0.1 to 1 micrometer. The cMUT transceiver 10 may include a gap that may be bounded by the lower electrode 12 and the diaphragm 16. The cavity 20 may be air or gas-filled or wholly or partially evacuated. However, in accordance with an exemplary embodiment of the present technique, a wholly or partially evacuated cavity 20 may be employed. Furthermore, the cavity 20 includes a dielectric floor 24. The cavity 20 may have a depth on the order of approximately tenths of a micron to a few microns.

According to an exemplary embodiment of the present technique, and as described further below, at least one element, such as a protruding element (e.g., FIGs. 1-5) or a receding element (e.g., FIG. 6), may be formed in the. cavity 20, and configured to adjust the gap, i.e., gap width, to be lower than the depth of the cavity 20, between the lower electrode 12 and the upper electrode 18, under certain modes of operation. Specifically, in a first exemplary embodiment, the at least one element may comprise a protruding element, such as a stud 22. The stud 22 may be disposed on the topside of the lower electrode 12. Alternatively, the stud 22 may be disposed on the bottom side of the diaphragm 16.

The stud 22 may comprise two layers. As depicted in the enlarged view of the stud in FIG. 1, the top layer of the stud 22 may comprise an insulating material such as a dielectric layer in order to prevent electrical shorting between the lower electrode 12 and the upper electrode 18. The dielectric layer may include materials such as, but not limited to, silicon oxide, silicon nitride, polymer and other non-conductive materials. Furthermore, the bottom layer of the stud 22 may comprise a conductive material, such as, but not limited to, metal, epi-silicon, single crystal silicon, polycrystalline silicon and other semi-conductor materials. The stud 22 may exhibit various shapes, such as, but not limited to circular, rectangular, and hexagonal. In addition, the stud 22 may be represented by a single stud, a ring shaped stud, hereinafter referred to as a ring stud, or any arrangement of studs, such as, but not limited to, an array of studs. Also, the sidewalls of the stud 22 may be vertical, tapered, or rounded.

Furthermore, the at least one element that may be formed in the cavity 20 of the cMUT transceiver 10 may be a receding element, such as a well 26. The well 26 may be etched in the cavity 20 (illustrated and discussed with reference to FIG. 6 below). Moreover, the cMUT transceiver 10 may include both the stud 22 and the well (not shown). Alternatively, a well may be etched on the lower electrode 12, and the stud 22 may be formed on the diaphragm 16. In accordance with yet another configuration, the studs 22 may be formed within a well.

Additionally, in accordance with a further aspect of the present technique, the cMUT transceiver 10 may include a source of bias potential (not shown), where the source of bias potential is configured to distend the diaphragm 16 towards the lower electrode 12. According to one embodiment of the present technique, the gap width between the lower electrode 12 and the upper electrode 18, may be varied by varying the height of the studs 22 and/or the depth of the wells, and by varying the bias potential based upon a mode of operation of the cMUT transceiver. While the cMUT transceiver 10 is operating as a transmitter, it may be beneficial to augment the depth of the cavity to facilitate larger deflection of the diaphragm to enhance the amplitude of the transmitted signal. However, when the cMUT transceiver is functioning as a receiver, it may be advantageous to have a smaller gap width between the lower electrode 12 and the upper electrode 18 in order to enhance the reception of signals. Consequently, the sensitivity of the cMUT transceiver 10 may be enhanced by adjusting the dimension of the gap between the lower electrode 12 and the upper electrode 18, thereby advantageously optimizing the performance of the cMUT transceiver 10 for transmitting and receiving signals.

As will be appreciated by one of ordinary skill in the art, the lower electrode 12 and the upper electrode 18 separated by the cavity 20 form a capacitance. For the cMUT transceiver 10 operating in the transmit mode as illustrated in FIG. 1, a large deflection of the diaphragm to increase the amplitude of the transmitted pulse, may be achieved by means of a deeper cavity 20. In the transmit mode, a smaller direct current (DC) bias permits a large alternating current (AC) excitation pulse to be applied which may advantageously result in a larger membrane deflection and a greater signal-to-noise ratio for the cMUT transceiver 10.

However, for the cMUT transceiver 10 operating in a receive mode, it may be desirable to have a smaller gap between the lower electrode 12 and the upper electrode 18 in order to enhance the sensitivity of the cMUT transceiver 10. FIG. 2 depicts a side view of a cross-section of a cMUT transceiver 10 operating in the receive mode. As illustrated in FIG. 2, the depth of the cavity 20 may be smaller than the depth of the cavity of the cMUT transceiver 10 of FIG. 1 operating in the transmit mode. This smaller cavity depth may result in a larger capacitance, which in turn may advantageously result in enhanced sensitivity of the cMUT transceiver 10. As depicted in FIG. 2, when the source of bias potential is applied to the cMUT transceiver 10, the diaphragm 16 may be deflected towards the lower electrode 12. However, due to the presence of the studs 22 in the cavity 20, the depth of the cavity 20 is significantly diminished. Therefore, deflection of the diaphragm 16 with diminished cavity depth may result in enhanced sensitivity of the cMUT transceiver 10 functioning as a receiver.

FIG. 3 is a top view of a cross-section of the cMUT transceiver 10 of FIG. 1 along the line 3-3. In the illustrated embodiment of FIG. 3, a ring stud is depicted. However, as described above, the stud may be in the form of a circle, a rectangle, a hexagon, or any other shape.

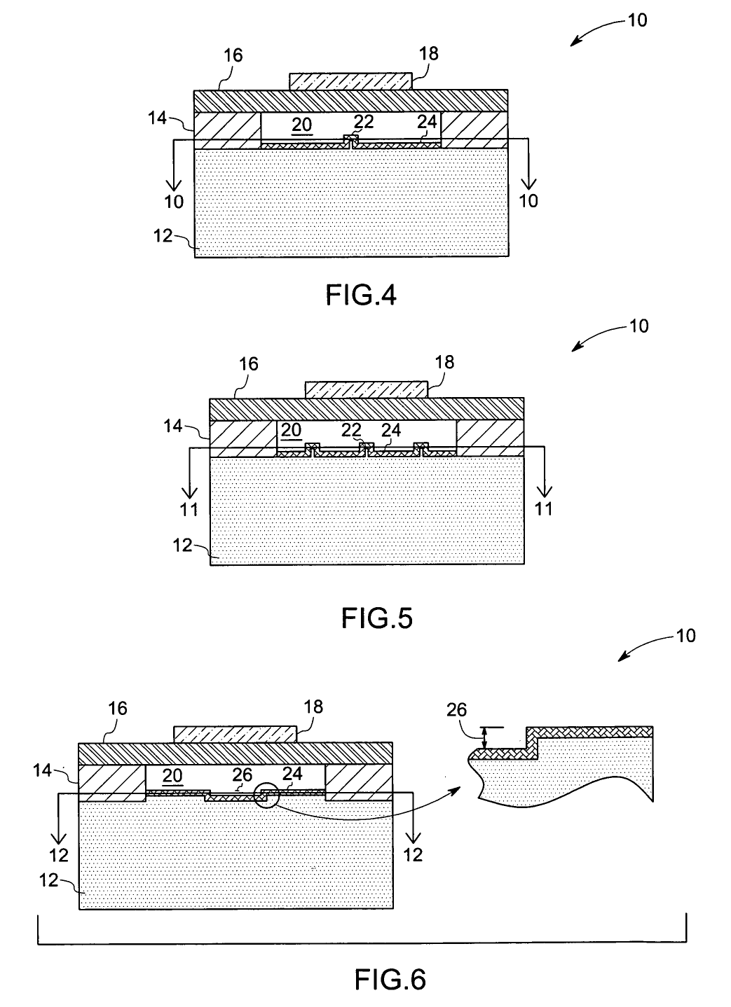

FIGS. 4-6 illustrate cross sectional views of alternate embodiments of the cMUT transceiver 10 operating in the transmit mode. With specific reference to FIG. 4, a cross-sectional side view of an alternate embodiment of the cMUT transceiver 10 operating in the transmit mode and having a single stud 22 disposed in the cavity 20 is illustrated. Furthermore, FIG. 5 illustrates yet another alternate embodiment of a cMUT transceiver 10, operating in the transmit mode and having a plurality of studs 22 arranged in an array that may be formed in the cavity 20. According to further aspects of the present technique, a receding element may be formed in the cavity 20. FIG. 6 illustrates an embodiment of the cMUT transceiver 10 operating in the transmit mode and having a receding element, such as a well 26, etched in the cavity 20.

Referring to FIG. 6, the well 26 may comprise two layers. As depicted in the enlarged view of the well in FIG. 6, the top layer of the well 26 may comprise an insulating material such as a dielectric layer in order to prevent electrical shorting between the lower electrode 12 and the upper electrode 18. The dielectric layer may include materials such as, but not limited to, silicon oxide, silicon nitride, polymer and other non-conductive materials. Furthermore, the bottom layer of the well 26 may comprise a conductive material, such as, but not limited to, metal, epi-silicon, single crystal silicon, polycrystalline silicon and other semi-conductor materials. The well 26 may exhibit various shapes, such as, but not limited to, circular, rectangular, and hexagonal. In addition, the well 26 may be represented by a single well, a ring shaped well, hereinafter referred to as a ring well, or any arrangement of wells, such as, but not limited to, an array of wells. Also, the sidewalls of the wells 26 may be vertical, tapered, or rounded.

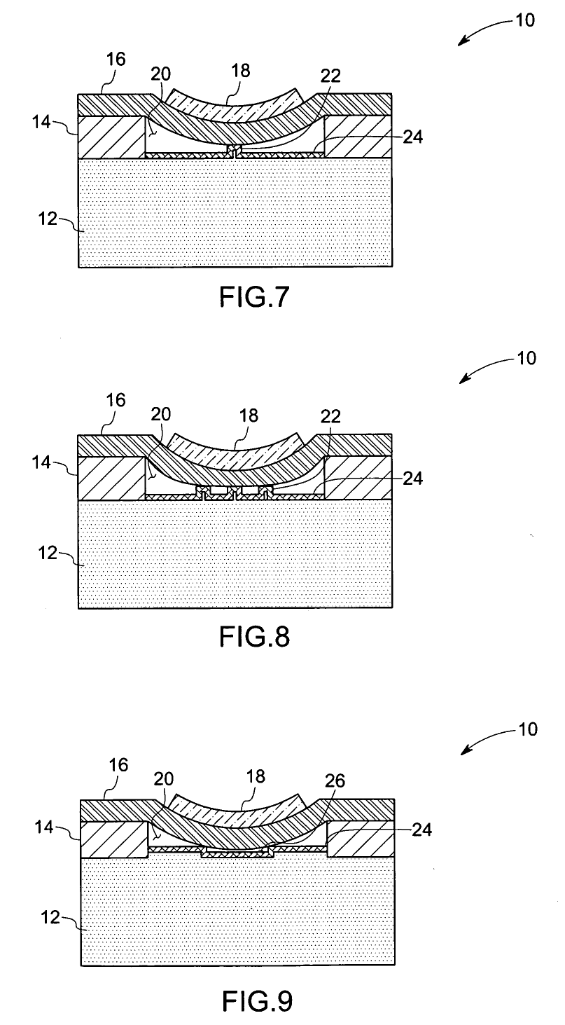

FIGS. 7-9 illustrate corresponding cross-sectional views of the cMUT transceiver 10 illustrated in FIGS. 4-6 operating in the receive mode. FIG. 7 depicts the cMUT transceiver 10 of FIG. 4 operating in the receive mode. Similarly, FIG. 8 illustrates the cMUT transceiver 10 of FIG. 5 operating in the receive mode. In a similar fashion, FIG. 9 illustrates the cMUT transceiver 10 of FIG. 6 functioning as a receiver.

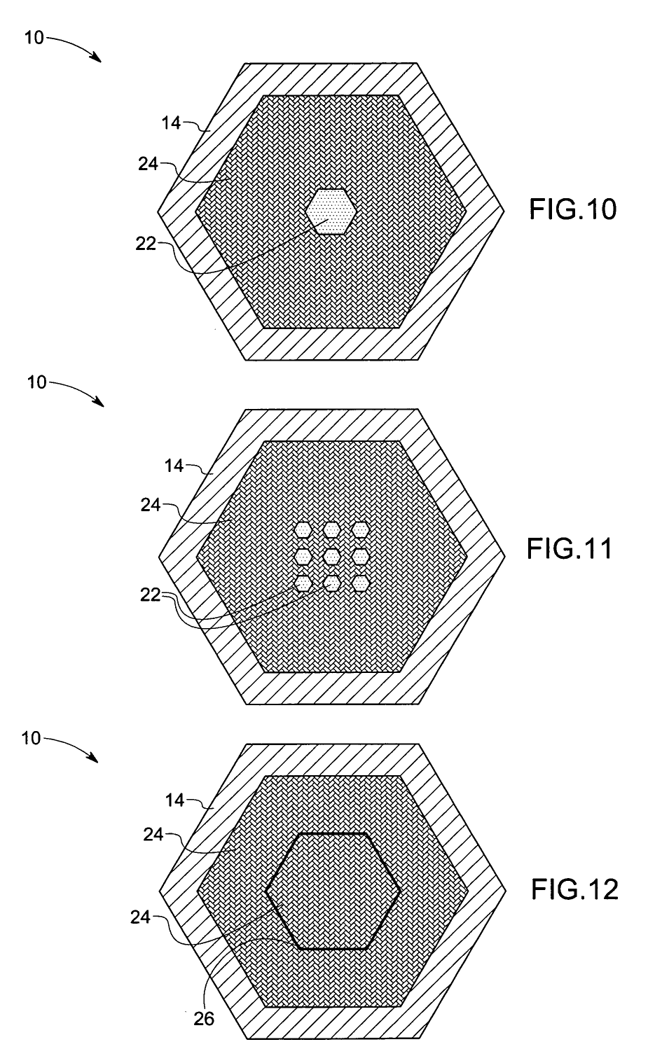

FIGS. 10-12 illustrate corresponding cross-sectional top views of the cMUT transceiver 10 illustrated in FIGS. 4-6. Referring specifically to FIG. 10, a top view of the cMUT transceiver 10 of FIG. 4 along line 10-10 and having a single stud 22 disposed in the cavity 20 of the cMUT transceiver 10 is illustrated. FIG. 11 illustrates a top view of the cMUT transceiver 10 of FIG. 5 along line 11-11, where an array of studs 22 is disposed in the cavity 20 of the cMUT transceiver 10. Similarly, a top view of the cMUT transceiver 10 of FIG. 6 along line 12-12 and having a well 26 etched in the cavity 20 of the cMUT transceiver 10 is illustrated.

The studs 22 and wells 26 may be implemented to vary the depth of the cavity 20 of the cMUT transceiver 10. Additionally, by varying the bias potential, the dimension of the gap between the lower electrode 12 and the upper electrode 18 may be optimized for transmitting and receiving signals. This optimization may be accomplished by employing a source of bias potential to control the deflection of the diaphragm 16 when the cMUT transceiver 10 is operating in the transmit and/or receive mode. For instance, when the cMUT transceiver is operating in the transmit mode, as illustrated in FIG. 1, a DC bias, lower than the collapse voltage, may be applied using the bias source, which may beneficially result in a large gap between the lower electrode 12 and the upper electrode 18 as depicted in FIG. 1. As will be appreciated by one skilled in the art, the collapse voltage is a bias voltage where the mechanical restoring force of the membrane deflection for small membrane deflections cannot balance the electrostatic force. The small DC bias enables a large AC excitation pulse to be applied that may result in a larger membrane deflection and a greater signal-to-noise ratio for the cMUT transceiver 10 operating in the transmit mode.

Furthermore, in the receive mode, a DC bias that is sufficient to collapse the diaphragm 16 onto the studs 22, may be applied via the source of bias potential. The applied voltage may deflect the diaphragm 16 onto the stud 22, as illustrated in FIG. 2. The reduced gap width between the lower electrode 12 and the upper electrode 18 may advantageously result in a greater capacitance change for a given incident acoustic wave, which in turn may lead to enhanced sensitivity of the cMUT transceiver 10. Additionally, the gap width between the lower electrode 12 and the upper electrode 18 in the cMUT transceiver 10 operating in the receive mode is smaller than in the cMUT transceiver 10 operating in the transmit mode. Moreover, while the bias voltage applied to the cMUT transceiver 10 functioning as a receiver is adequate to attract the upper electrode 18 onto the stud 22, the bias potential may be lower than the collapse voltage for the lower electrode 12 and the upper electrode 18.

As discussed above, the studs 22 may protrude from the floor of the cavity 20. Hence, the effective depth of the cavity 20 between the top of the studs 22 and the upper electrode 18 (i.e., the “gap”) may be smaller thereby necessitating a smaller bias potential to collapse the diaphragm 16 onto the studs 22. In one exemplary embodiment, the height of the studs 22 may be less than 0.2 micrometers, for example. Moreover, the studs may be disposed on the lower electrode 12 or on the upper electrode 18. The depth of the cavity 20 of the cMUT transceiver 10 functioning as a receiver may be regulated by the height of the stud 22 when the diaphragm 16 is collapsed onto the studs 22. This smaller cavity depth may advantageously result in a larger capacitance change for a given incident ultrasound wave and thus may result in enhanced sensitivity of the cMUT transceiver 10 operating in the receive mode.

In accordance with an exemplary embodiment of the present invention, a cMUT transceiver 10 where the gap between the lower electrode 12 and the upper electrode 18 may be adjusted by implementing studs and/or wells, and by varying the bias potential was described. In accordance with the present exemplary embodiments, the cMUT transceiver 10 may be optimized for performance as both a transmitter and a receiver. Similar principles may be employed to configurations with separate transmit and receive cells thereby enabling discrete optimization of the cMUT cells functioning as transmitters and receivers, as described further below.

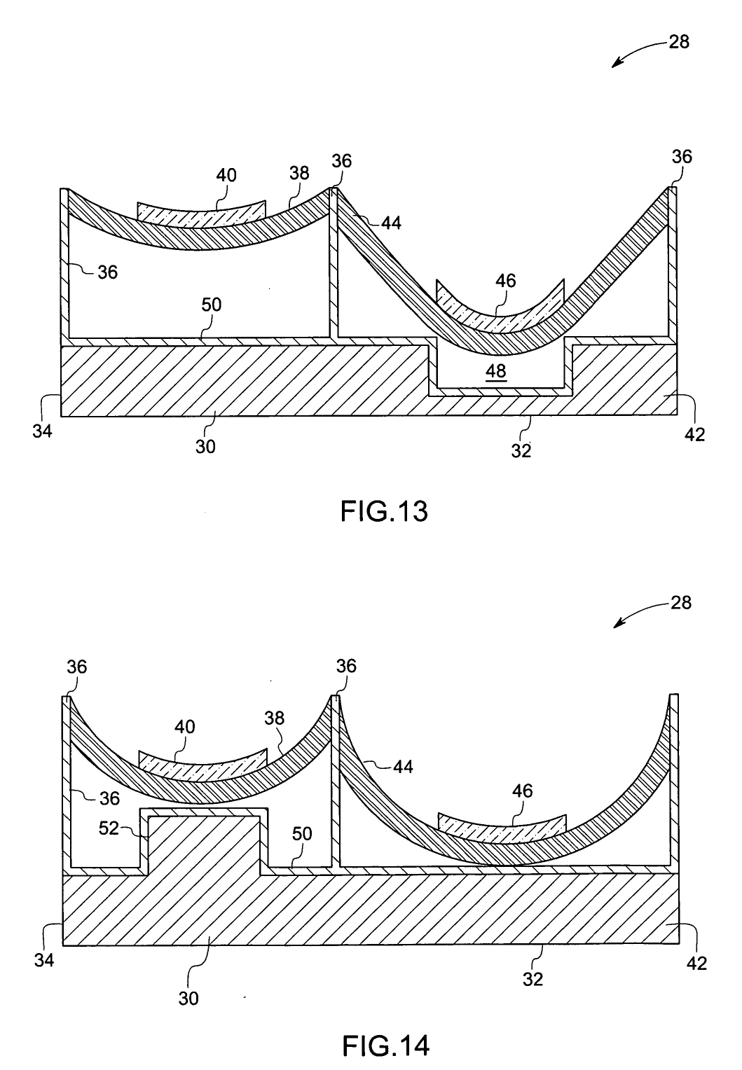

FIGS. 13 and 14 illustrate alternate embodiments of a dual cavity cMUT unit cell 28 having distinct transmitter and receiver cell structures such that gaps having different depths may be implemented in each of the transmit and receive modes. In a presently contemplated configuration, the cMUT unit cell 28 depicted in FIGS. 13 and 14 includes a first cell (receiver cell 30), which is configured to operate in a receive mode. As described further below, the receiver cell 30 includes a lower electrode and an upper electrode and a gap having a first gap width. In addition, the cMUT unit cell 28 includes a second cell (transmitter cell 32) that is configured to operate in a transmit mode. As with the receiver cell 30, the transmitter cell 32 also includes a lower electrode and an upper electrode, and a gap having a second gap width larger than the first gap width, as described further below.

Referring initially to FIG. 13, the receiver cell 30 includes a lower electrode 34. A plurality of support posts 36 may be disposed on the lower electrode 34. Moreover, a diaphragm 38 may be disposed on the plurality of support posts 36. In addition, an upper electrode 40 may be disposed on the diaphragm 38. The receiving cell 30 has a gap having a first gap width between the lower electrode 34 and the upper electrode 40. The first gap width may be configured to optimize the change in capacitance for a given incident ultrasound signal when the cMUT unit cell 28 is operating in the receive mode.

The cMUT unit cell 28 further includes a transmitter cell 32, which may be disposed adjacent to the receiver cell 30, may include a lower electrode 42. Alternatively, the transmitter cell 32 may also be disposed isolated from the receiver cell 30. As with the receiver cell 30, the transmitter cell 32 further comprises a plurality of support posts 36 disposed on the lower electrode 42. In addition a diaphragm 44 may be disposed on the plurality of support posts 36 and an upper electrode 46 may be disposed on the diaphragm 44. Furthermore, according to the present exemplary embodiment, the transmitter cell 32 may include a micromachined well 48. The presence of the well 48 provides a gap having a larger gap width between the transmitting lower electrode 42 and the transmitting upper electrode 46 when compared to the gap width of the receiver cell 30, which may in turn facilitate enhanced displacement of the transmitting diaphragm 44 when the cMUT unit cell 28 is operating in the transmit mode. Consequently, an ultrasound wave of enhanced amplitude may be achieved when the cMUT unit cell 28 is operating in the transmit mode. Moreover, an insulation layer 50 may be disposed on the receiving lower electrode 34, the transmitting lower electrode 42 and the floor of the well 48.

Further, while the present exemplary embodiment depicted in FIG. 13 illustrates a well 48 formed in the transmitting lower electrode 42 to provide varied gap widths in each of the receiver cell 30 and the transmitter cell 32, in an alternate exemplary embodiment depicted in FIG. 14, a protruding element, such as a stud 52, may be disposed on the receiving lower electrode 34. The stud 52 may be configured to reduce the gap width between the receiving lower electrode 34 and the receiving upper electrode 40, thereby optimizing the change in capacitance for a given incident ultrasound wave. Furthermore, an insulating layer may be disposed on the stud 52. The insulating layer may also be disposed on the receiving lower electrode 34. Alternately, the dual cavity cMUT unit cell 28 may be configured to include each of a well 48 in the transmitter cell 32 and a stud 52 in the receiver cell 30 or any combinations of studs and wells thereof.

In the exemplary embodiment of the dual cavity cMUT unit cells 28 illustrated in FIGS. 13-14, the lateral dimensions of the receiver and transmitter cells may be different. This facilitates the application of the dual cavity cMUT unit cell 28 in various fields. For example, the dual cavity cMUT unit cells 28 may find application in harmonic imaging, where the operating frequency of the receiver cell 30 and the transmitter cell 32 may be advantageously tailored by adjusting the respective sizes of each of the cells. The dual cavity unit cell 28 may be of the same size as the cMUT transceiver 10 of FIG. 1. As will be appreciated by one of ordinary skill in the art, by separating the cMUT cells based on their functionality, that is transmitting and receiving, a sensing area including a plurality of distinct transmitter and receiver cMUT cells may experience a signal loss due to a reduction in the sensing area while either of the transmitter cells or the receiver cells are operational. However, separating the structure into distinct cells for transmitting and receiving, as depicted in FIGS. 13-14, may more than compensate for the loss of signal incurred when only one set of cMUT cells, that is, either the transmitter cells or the receiver cells, are operational. The transmitter and receiver cells may now be separately optimized thereby resulting in enhancement of sensitivity that may exceed the loss in active sensing area.

Moreover, as described with regard to the cMUT transceiver 10, the dual cavity cMUT unit cell 28 may include at least one source of bias potential, where the source of bias potential is configured to distend the receiving diaphragm 38 and the transmitting diaphragm 44 towards their corresponding lower electrodes 34 and 42.

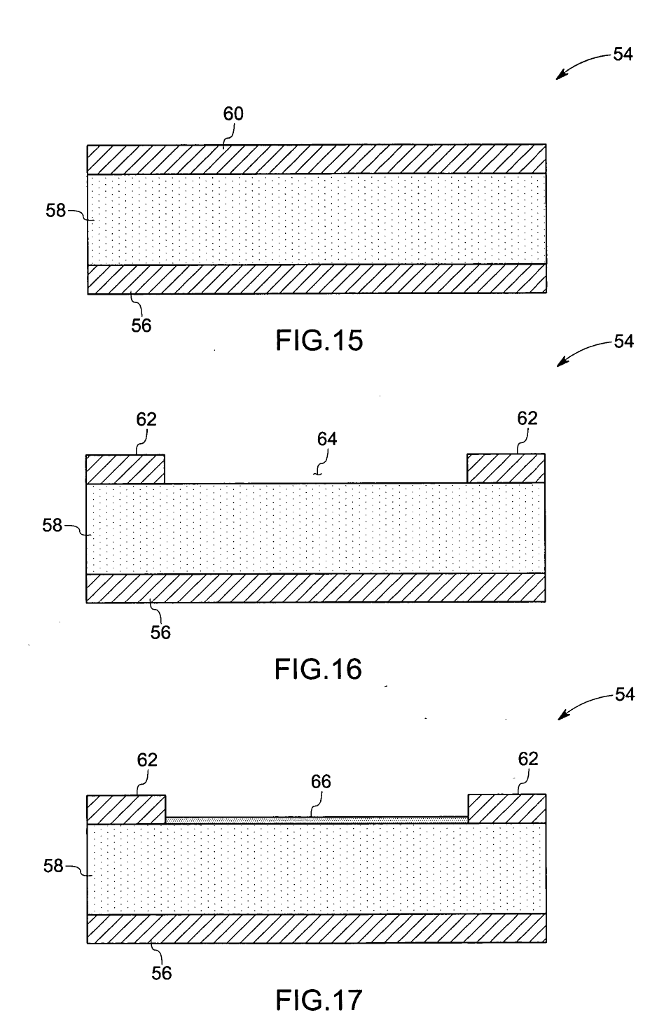

According to further aspects of the present technique, a method for fabricating one embodiment of a cMUT transceiver is presented. FIGS. 15-20 depict a process flow for fabricating the cMUT transceiver, where the studs may be disposed on the diaphragm. FIG. 15 illustrates an initial step in the process of fabricating a bottom portion 54 (a low resistivity prime wafer), which may include a lower electrode of a cMUT transceiver. As depicted in FIG. 15, a first oxide layer 56 and a second oxide layer 60 may be formed by means of an oxidation process that may be a dry oxidation process, a wet oxidation process, or a combination of the two, on opposing sides of a substrate such as a high-conductivity silicon layer 58. The second oxide layer 60 defines the gap between the lower electrode and the upper electrode. As illustrated in FIG. 16, lithography and wet etching may be employed to etch away a section of the second oxide layer, thereby defining a plurality of support posts 62 and a cavity 64 that may be defined by the support posts. Subsequently, as depicted in FIG. 17, a oxidation process may be employed to provide electrical insulation 66 in the cavity 64.

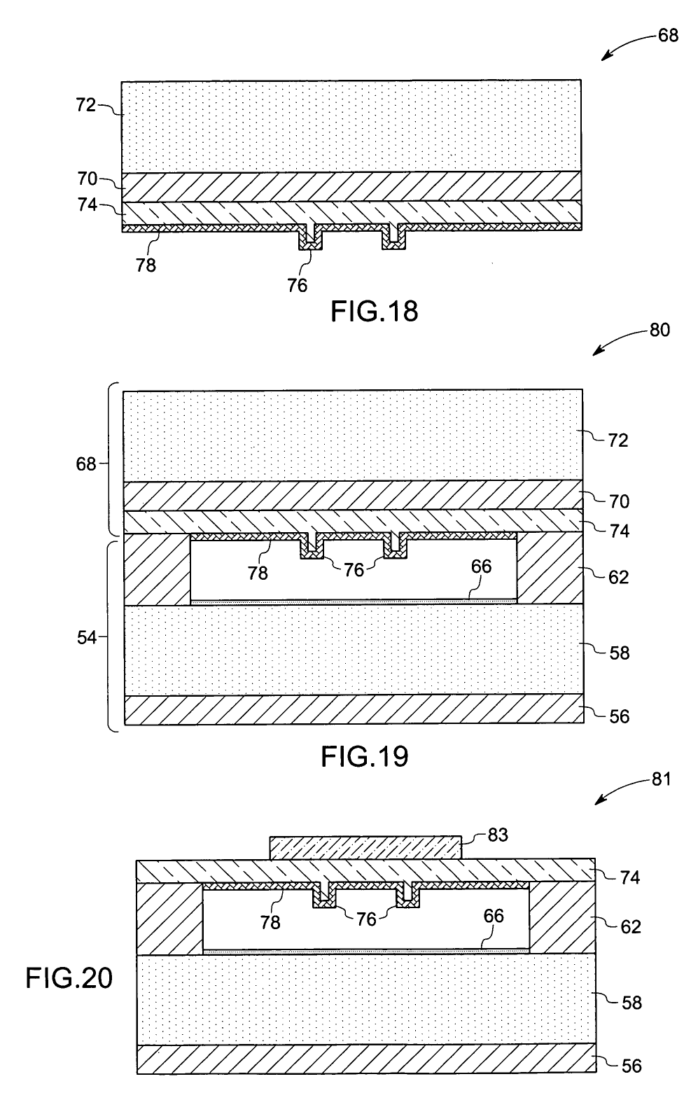

The method for fabricating a cMUT transceiver further comprises fabricating a top portion 68 (a Silicon on Insulator (SOI) wafer) that may include an upper electrode. Alternatively, as will be appreciated by one skilled in the art, a pre-fabricated SOI including a silicon substrate, a buried oxide layer and a silicon handle wafer may be employed in the fabrication of the cMUT transceiver. As illustrated in FIG. 18, the top portion 68 includes a buried oxide (“box”) layer 70 that may be disposed on a handle wafer 72. In addition, a conductive or low resistivity layer, such as, but not limited to, an epi-silicon, single crystal silicon, and polycrystalline silicon layer, may be disposed on the oxide box layer 70, where the conductive layer may be configured to function as a diaphragm 74. Alternatively, a non-conductive or high resistivity layer, such as, but not limited to, an epi-silicon, single crystal silicon, and polycrystalline silicon layer, may be disposed on the oxide box layer 70, where this layer may be configured to function as a diaphragm 74. Moreover, at least one element, such as a protruding stud 76, may be formed on the diaphragm 74. The stud 76 may be formed by employing a lithography process followed by a dry etching procedure, which may be followed by a thermal oxidation process to provide an insulating layer 78 on the studs 76. As will be appreciated by one skilled in the art, techniques such as, but not limited to, plasma enhanced chemical vapor deposition (PECVD) and low-pressure chemical vapor deposition (LPCVD) may also be employed to form the studs 76. Alternatively, the studs 76 may be formed by the deposition of a material, such as a metal, on the diaphragm 74, followed by a dielectric deposition process to provide an insulating layer on the studs 76. Furthermore, the height of the studs 76 formed may be configured to define the gap width within the cavity 64 between the upper electrode and the lower electrode when the cMUT transceiver is functioning in a receive mode.

Furthermore, as depicted in FIG. 19, a structure 80 may now be formed by disposing the top portion 68 (SOI wafer) on the bottom portion 54 (prime wafer) by means of fusion bonding between the SOI wafer and the prime wafer 54. Mechanical polishing or grinding followed by wet etching with chemicals such as, but not limited to, tetramethyl ammonium hydroxide (TMAH), potassium hydroxide (KOH), and Ethylene Diamine Pyrocatechol (EDP) may be employed to remove the handle wafer 72. Following removal of the handle wafer 72, the oxide box layer 70 may be removed by buffered hydrofluoric acid (BHF). Subsequently, as illustrated in FIG. 20, an upper electrode 83 may be disposed on the diaphragm 74 to form the cMUT transceiver 81. FIG. 20 illustrates a cMUT transceiver 81 where the studs 76 are disposed on the diaphragm 74. Moreover, as will be appreciated by one of ordinary skill, surface micromachining may also be employed to include studs and/or wells. With surface micromachining, the diaphragm is deposited, instead of being bonded from an SOI wafer as in the bulk micromachining process. This may be followed by the removal of any sacrificial layers underneath the diaphragm (such as the oxide), sealing the cavity with a vacuum, and deposition of the top electrode.

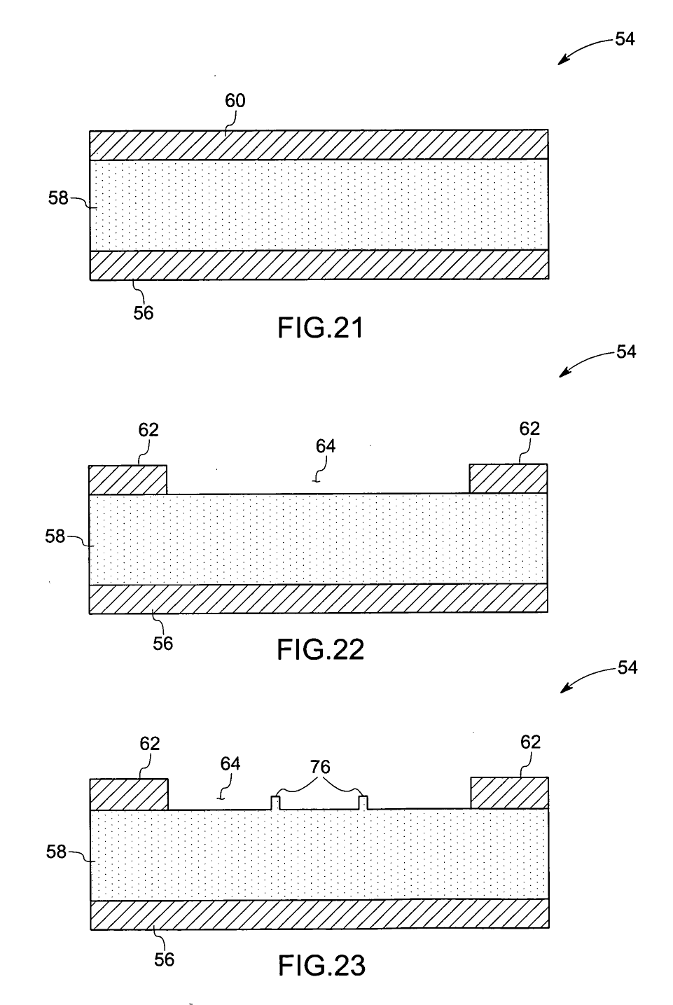

The process flow described with reference to FIGS. 15-20 depicts the process for fabricating the cMUT transceiver where the studs 76 may be disposed on the diaphragm 74. As will be appreciated by one skilled in the art, similar techniques may also be employed to fabricate a dual cavity cMUT unit cell structure. In a similar fashion, FIGS. 21-26 depict a process flow for fabricating a cMUT transceiver where the studs may be disposed on the lower electrode as will be described below, and as previously illustrated in FIG. 1.

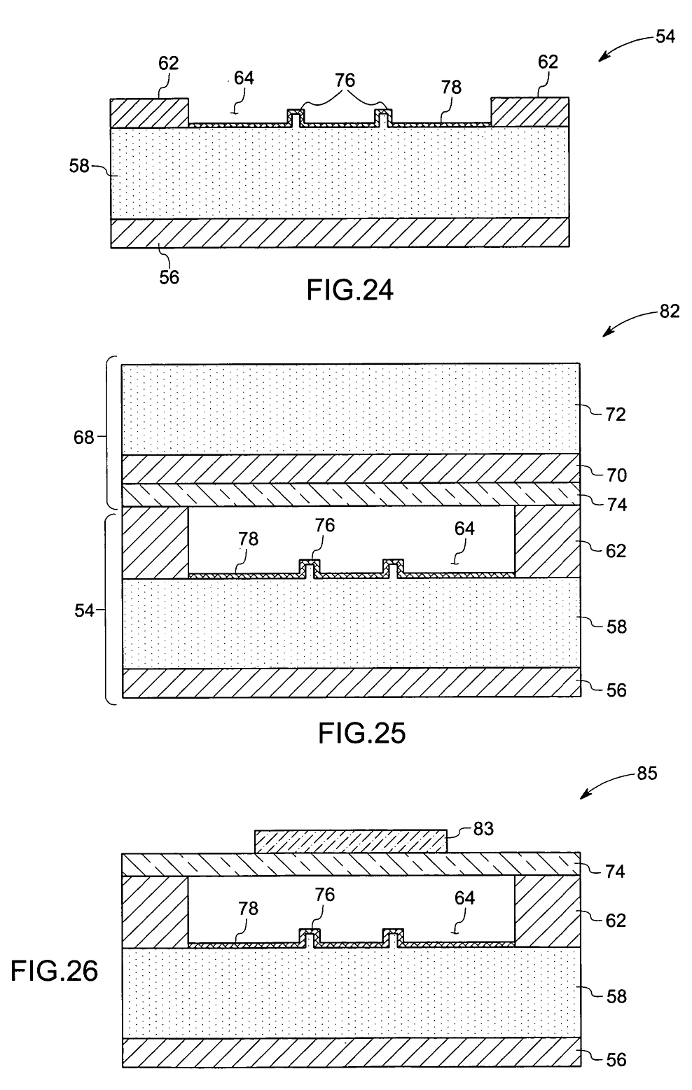

FIG. 21 illustrates an initial step in the process of fabricating a bottom portion 54 (a low resistivity prime wafer) of a cMUT transceiver, where a first oxide layer 56 and a second oxide layer 60 are fabricated by means of an oxidation process, such as, but not limited to, a dry oxidation process, a wet oxidation process, or a combination of the two, and may be disposed on a high-conductivity silicon layer 58. The second oxide layer 60 defines the gap width between the lower electrode and the upper electrode. As illustrated in FIG. 22, lithography and wet etching may be employed to etch away a section of the second oxide layer, thereby defining a plurality of support posts 62 and a cavity 64 that may be defined by the support posts. Subsequently, as depicted in FIG. 23, a lithography process that may be followed by a etching process may be employed to form the studs 76 in the cavity 64. As depicted in FIG. 24, an oxidation process, to provide an electrical insulation layer 78 on the studs 76, may follow the formation of the studs 76. Alternatively, as described hereinabove, the studs 76 may be formed by the deposition of a material, such as a metal, on the lower electrode 54, followed by an dielectric deposition process to provide an insulating layer on the studs 76.

FIG. 25 illustrates an alternate embodiment of the present technique where the studs 76 are disposed in the cavity 64. The present exemplary method for fabricating a cMUT transceiver further includes fabricating a top portion 68 (SOI wafer). Alternatively, as will be appreciated by one skilled in the art, a pre-fabricated SOI including a silicon substrate, a buried oxide layer and a silicon handle wafer may be employed in the fabrication of the cMUT transceiver. As illustrated in FIG. 25, the top portion 68 includes an oxide box layer 70 disposed on a handle wafer 72. In addition, a conductive or low resistivity layer, such as, but not limited to, an epi-silicon, single crystal silicon, or polycrystalline silicon layer, may be disposed on the oxide box layer 70, where this layer may be configured to function as a diaphragm 74. Alternatively, a non-conductive or high resistivity layer, such as, but not limited to, an epi-silicon, single crystal silicon, and polycrystalline silicon layer, may be disposed on the oxide box layer 70, where this layer may be configured to function as the diaphragm 74.

Furthermore, as depicted in FIG. 25, a structure 82 may now be formed by disposing the top portion 68 on the bottom portion 54 by means of fusion bonding between the SOI wafer and the prime wafer. The handle wafer 72 may be removed by mechanical polishing or grinding followed by wet etching with etchants such as, but not limited to, TMAH, KOH, and EDP. Following removal of the handle wafer, the oxide box layer 70 may be removed by BHF. Subsequently, as illustrated in FIG. 26, the upper electrode 83 may be disposed on the diaphragm 74 to form the cMUT transceiver 85. FIG. 26 illustrates a cMUT transceiver 85 where the studs 76 are disposed on the lower electrode 58. Moreover, as will be appreciated by one of ordinary skill in the art, surface micromachining may also be employed to include studs and/or wells. With surface micromachining, the diaphragm is deposited, instead of being bonded from an SOI wafer as in the bulk micromachining process. This may be followed by the removal of any sacrificial layers underneath the diaphragm, such as the oxide, sealing the cavity with a vacuum, and deposition of the top electrode.

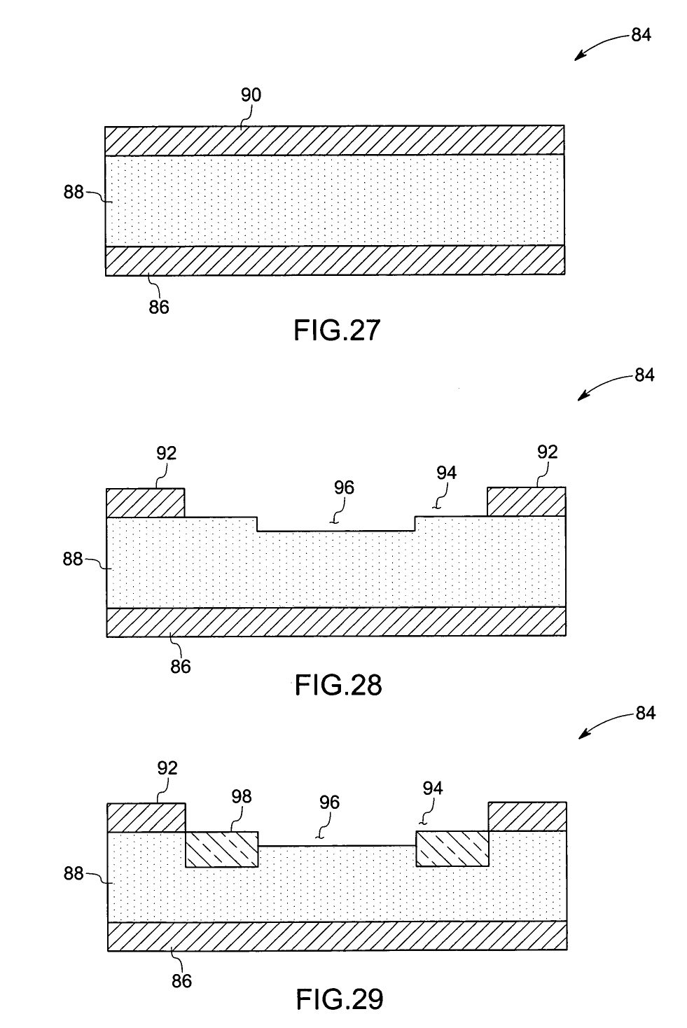

The process flows described hereinabove describe the process for forming studs in the cavity of a cMUT transceiver. As previously mentioned, similar techniques may also be employed to fabricate a dual cavity cMUT unit cell structure. As will be appreciated by those skilled in the art, similar processes may be followed for etching a receding element, such as a well, in the cavity of the cMUT transceiver, as described further below with reference to FIGS. 27-32. This concept allows the diaphragm to collapse onto a protruding, heavily doped region while maintaining a desirable thin gap at receive mode for improved sensitivity. As will be appreciated by one skilled in the art, similar techniques may also be employed to fabricate a dual cavity cMUT unit cell structure.

FIGS. 27-32 depict an exemplary process flow, according to aspects of the present technique, for fabricating a cMUT cell where a receding element such as a well is formed in a gap between a lower electrode and an upper electrode. FIG. 27 illustrates an initial step in the process of fabricating a bottom portion 84 (prime wafer) of a cMUT cell that may include a lower electrode, where a first oxide layer 86 and a second oxide layer 90 are fabricated by means of an oxidation process, such as, but not limited to, a dry oxidation process, a wet oxidation process, or a combination of the two, and may be disposed on a low-conductivity silicon layer 88.

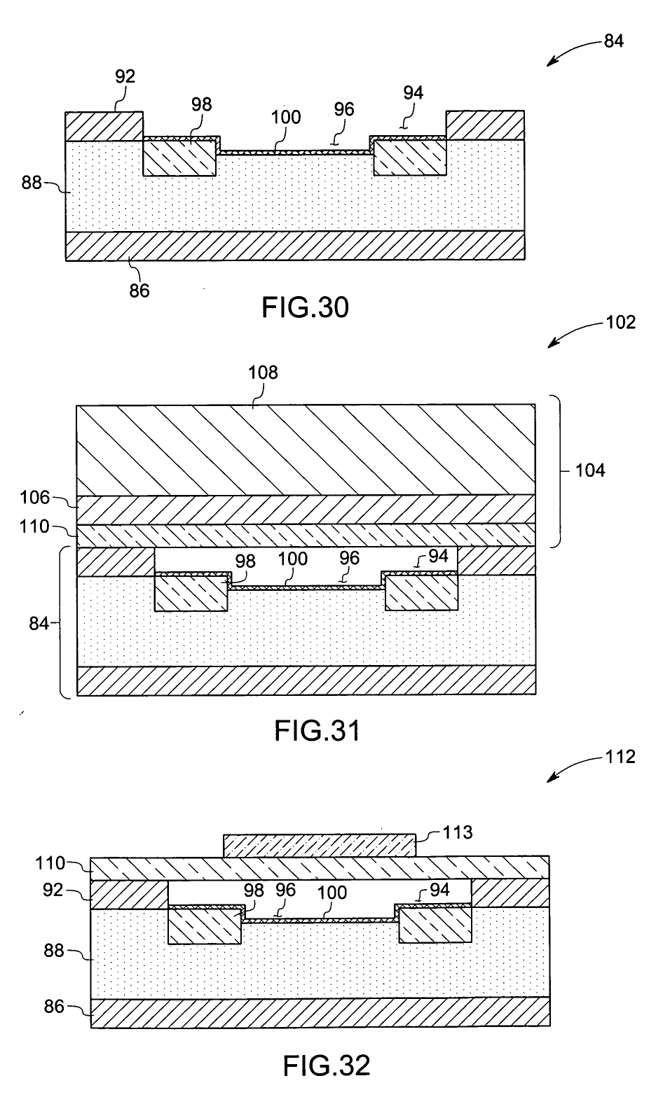

As illustrated in FIG. 28, a first lithography and etching process may be employed to etch away a section of the second oxide layer 90, thereby defining a plurality of support posts 92 and a cavity 94 that may be defined by the support posts 92. Additionally, as depicted in FIG. 28, a second lithography and etching step may be employed to define a receding element, such as a well 96, formed at the bottom of the cavity 94. In this embodiment, the silicon layer 88 may be heavily doped, as mentioned previously. Alternatively, as depicted in FIG. 29, the lightly doped silicon layer 88 of FIG. 27 may be heavily doped in regions adjacent to the support posts 92. These heavily doped regions referenced by numeral 98 may be incorporated through an additional doping step. The heavily doped regions 98 can also be applied to the protruding element, such as studs, as discussed in previous sections. Subsequent to the doping step, an oxidation process may provide electrical insulation 100, as illustrated in FIG. 30.

Additionally, the method for fabricating a cMUT cell further comprises fabricating a top portion 104 (SOI wafer) as described above. Alternatively, as will be appreciated by one skilled in the art, a pre-fabricated SOI including a silicon substrate, a buried oxide layer and a silicon handle wafer may be employed in the fabrication of the cMUT transceiver. As illustrated in FIG. 31, the top portion 104 may include an oxide box layer 106 having a first side and a second side and may be disposed on a handle wafer 108. In addition, a conductive or low resistivity layer, such as, but not limited to, an epi-silicon, single crystal silicon, or polycrystalline silicon layer, may be disposed on a second side of the oxide box layer 106, where this layer may be configured to function as a diaphragm 110. Alternatively, a non-conductive or high resistivity layer, such as, but not limited to, an epi-silicon, single crystal silicon, and polycrystalline silicon layer, may be disposed on the second side of the oxide box layer 106, where this layer may be configured to function as a diaphragm 110.

Furthermore, as depicted in FIG. 31, a structure 102 may now be formed by disposing the top portion 104 on the bottom portion 84 by means of fusion bonding between the SOI wafer and the prime wafer. The handle wafer 108 can be removed by mechanical polishing or grinding followed by wet etching with etchants such as, but not limited to TMAH, KOH, and EDP. The oxide box layer 106 may be removed by BHF. Subsequently, as illustrated in FIG. 31, the upper electrode 113 may be disposed on the diaphragm 110 to form the cMUT transceiver 112. FIG. 32 illustrates a cMUT transceiver 112 where the well 96 is disposed on the lower electrode 88. Moreover, as will be appreciated by one of ordinary skill in the art, surface micromachining may also be employed to include studs and/or wells. With surface micromachining, the diaphragm is deposited, instead of being bonded from an SOI wafer as in the bulk micromachining process. This may be followed by the removal of any sacrificial layers underneath the diaphragm, (such as the oxide), sealing the cavity with a vacuum, and deposition of the top electrodes.

The process flow described hereinabove describes the process for forming a well in the cavity of the cMUT cell 112. Similar processes may be followed for forming a protruding element, such as a stud, in the cavity of a cMUT cell 112. However, in accordance with an exemplary embodiment of the present technique, it may be desirable that the heavily doped regions reside in the silicon layer of the studs in order for the diaphragm to be preferentially attracted to the stud regions, resulting in a diminished gap width for improved receive mode operation.

As previously described, in accordance with further embodiments of the present techniques, a dual cavity unit cell structure, such as the dual cavity unit cells illustrated in FIGS. 13 and 14, may be implemented. As previously described, the dual cavity unit cell structure includes a first cell that may be configured to operate as a receiver. Additionally, the dual cavity cMUT unit cell structure may include a second cell that may be configured to operate as a transmitter. In accordance with further aspects of the present technique, an exemplary method for fabricating a dual cavity cMUT cell unit structure is described. As previously mentioned, exemplary methods described with reference to FIGS. 15-32 may be employed to fabricate the dual cavity cMUT cell unit structure. The method includes fabricating a first cell that may be configured to operate as a receiver where the receiving cMUT cell may include a lower electrode and an upper electrode. Furthermore, the method may entail the fabrication of a second cell that may be configured to operate in a transmitting mode, where the transmitter cMUT cell may include a lower electrode and an upper electrode. Furthermore, the method may entail the formation of one of a protruding element and a receding element in one of the first cell and the second cell.

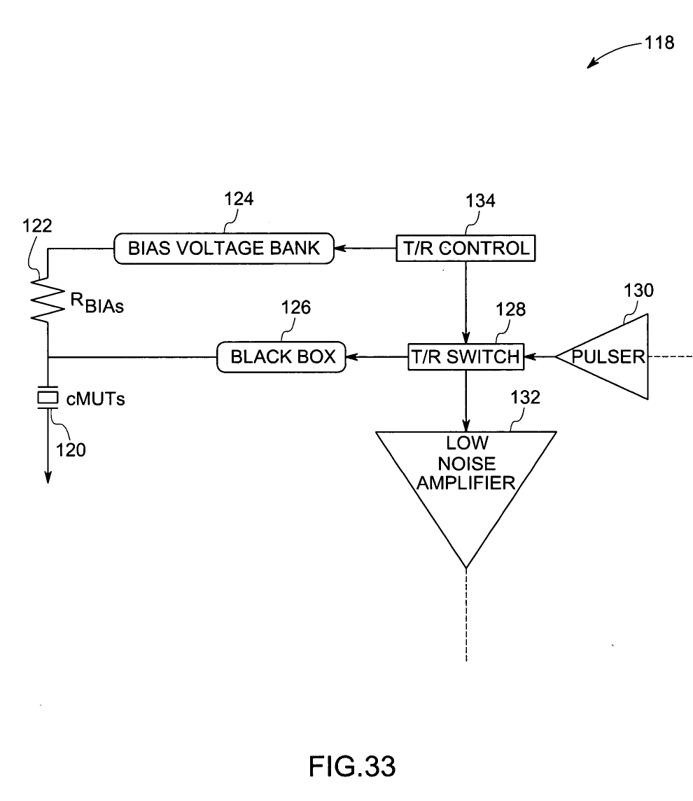

FIG. 33 is a block diagram of a cMUT transceiver system 118 that may include exemplary cMUT cells 120 fabricated in accordance with aspects of the present technique. The system 118 may include a bank of resistors 122 and may be coupled to the cMUT cells 120. In addition, the system 118 may include a bias voltage bank 124 that may be coupled to the resistors 122, which may be powered by at least one external voltage. Moreover, DC-to-DC converters that may be present in the bias voltage bank may generate various pre-determined bias voltages, which may be discrete or continuous. The bias voltage bank 124 may be implemented by employing discrete electronic devices disposed on a board. Alternatively, the bias voltage bank may be implemented as an application specific integrated circuit (ASIC). By implementing an ASIC to integrate the bias voltage bank 124 and the remainder of the functional blocks it may be possible to achieve System-on-Chip (SOC).

The black box 126 may comprise multiplexer circuits and may be coupled to the resistors 122. The transmit/receive (T/R) switch 128 that may be coupled to the black box 126 may typically include switch circuits and may be designed to switch between transmitting and receiving signals. Furthermore, the system 118 may include a pulser 130 that may be coupled to the T/R switch 128 may be utilized to generate the AC excitation pulses. The low noise amplifier (LNA) 132 that may be coupled to the T/R switch 128 may be employed to enhance signals. Additionally, in accordance with an exemplary embodiment of the present technique, a T/R Control block 134 that may be coupled to the T/R switch 128 may be employed to coordinate the functioning of the bias voltage bank 124 and the T/R switch 128. Programmable devices, such as, but not limited to, field programmable gate arrays (FPGA) and logic circuits, may be utilized to implement the T/R Control 134. Off-the-shelf parts may be utilized to implement the pulser 130 and the LNA 132.

While operating the cMUT transceivers 120 in a transmit mode, a DC bias voltage provided by the bias voltage bank 124 and an AC excitation pulse that has been generated by the pulser may be applied to the cMUT transceivers 120. The T/R control 134 may be utilized to set the bias voltage bank 124 and the T/R switch 128 to the transmit mode to enable feeding the DC bias voltage and ultrasound pulses to the cMUTs 120. These ultrasound pulses may be transformed into acoustic signals by means of the cMUTs 120.

While operating in a receive mode, a larger DC bias voltage provided by the bias voltage bank 124 may be applied to the cMUTs 120. The T/R control 134 may be employed to set the bias voltage bank 124 and the T/R switch 128 to the receive mode. Upon receiving reflected acoustic signals, the cMUTs 120 may transform these acoustic signals to electrical signals. Furthermore, these electrical signals are channeled to the LNA 132 for signal amplification.

According to an aspect of the present technique, a cMUT transceiver is presented. As described hereinabove with reference to the figures, the cMUT transceiver may include a lower electrode. Furthermore, a diaphragm may be disposed adjacent to the lower electrode such that a gap, having a first gap width, is formed between the diaphragm and the lower electrode. In addition, according to aspects of the present technique, at least one element may be formed in the gap. The element is arranged to provide a second gap width between the diaphragm and the lower electrode. In one embodiment, the first gap width is greater than the second gap width. Furthermore, the element may include a protruding element such as a stud. The element may further include a receding element such as a well. The cMUT transceiver may include an upper electrode coupled to the diaphragm. In addition, the cMUT transceiver may include a source of bias potential that may be employed to distend the diaphragm towards the lower electrode during operation of the cMUT transceiver.

The cMUT transceivers 10 and the method of fabricating the cMUT transceivers described hereinabove enable the fabrication of cMUT transceivers with enhanced sensitivity. The performance of the cMUT transceiver while operating both as a transmitter and a receiver may be advantageously enhanced. These cMUT transceivers may find application in various fields such as medical imaging, non-destructive evaluation, wireless communications, security applications, gas sensing, and other applications.

Furthermore, dual cavity cMUT unit cells 28 and the method of fabricating the dual cavity cMUT unit cells described hereinabove facilitate the optimization of operation of separate cells for transmitting and receiving signals, which may result in enhanced sensitivity of the dual cavity cMUT unit cells. These dual cavity cMUT unit cells may find application in fields such as medical imaging, non-destructive evaluation, wireless communications, security applications, gas sensing, and other applications.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A capacitive micromachined ultrasound transducer cell comprising:
a lower electrode;
a diaphragm disposed adjacent to the lower electrode such that a gap having a first gap width is formed between the diaphragm and the lower electrode; and
at least one element formed in the gap, wherein the at least one element is arranged to provide a second gap width between the diaphragm and the lower electrode.

2. The capacitive micromachined ultrasound transducer cell of claim 1, wherein the at least one element comprises a protruding element.

3. The capacitive micromachined ultrasound transducer cell of claim 2, wherein the protruding element comprises a stud.

4. The capacitive micromachined ultrasound transducer cell of claim 1, wherein the at least one element comprises a receding element.

5. The capacitive micromachined ultrasound transducer cell of claim 4, wherein the receding element comprises a well.

6. The capacitive micromachined ultrasound transducer cell of claim 1, wherein the first gap width is greater than the second gap width.

7. The capacitive micromachined ultrasound transducer cell of claim 1, further comprising a source of bias potential, wherein the source of bias potential is configured to distend the diaphragm towards the lower electrode.

8. The capacitive micromachined ultrasound transducer cell of claim 1, further comprising an upper electrode coupled to the diaphragm.

9. A capacitive micromachined ultrasound transducer cell comprising:
a lower electrode comprising a topside and a bottom side;
a plurality of support posts disposed on the topside of the lower electrode and configured to define a cavity;
a diaphragm disposed on the plurality of support posts to provide a gap bounded by the diaphragm and the lower electrode;
an upper electrode disposed on the diaphragm; and
at least one element formed in the cavity and configured to provide a gap width between the lower electrode and the upper electrode, which is less than the depth of the cavity.

10. The capacitive micromachined ultrasound transducer cell of claim 9, further comprising a source of bias potential, wherein the source of bias potential is configured to distend the diaphragm towards the lower electrode.

11. The capacitive micromachined ultrasound transducer cell of claim 10, wherein the gap width between the lower electrode and the upper electrode is adjusted by altering the bias potential and a height of at least one element formed in the cavity based upon a mode of operation of the cell.

12. The capacitive micromachined ultrasound transducer cell of claim 11, wherein the mode of operation of the cell is a transmit mode.

13. The capacitive micromachined ultrasound transducer cell of claim 11, wherein the mode of operation of the cell is a receive mode.

14. The capacitive micromachined ultrasound transducer cell of claim 9, wherein the at least one element formed in the cavity is a protruding element.

15. The capacitive micromachined ultrasound transducer cell of claim 14, wherein the protruding element comprises a stud.

16. The capacitive micromachined ultrasound transducer cell of claim 15, wherein the stud is disposed in the cavity on the topside of the lower electrode.

17. The capacitive micromachined ultrasound transducer cell of claim 15, wherein the stud is disposed on a bottom side of the diaphragm.

18. The capacitive micromachined ultrasound transducer cell of claim 15, wherein the stud exhibits a circular shape.

19. The capacitive micromachined ultrasound transducer cell of claim 15, wherein the stud exhibits a rectangular shape.

20. The capacitive micromachined ultrasound transducer cell of claim 15, wherein the stud exhibits a hexagonal shape.

21. The capacitive micromachined ultrasound transducer cell of claim 15, wherein the stud comprises a ring stud.

22. The capacitive micromachined ultrasound transducer cell of claim 15, wherein the stud comprises an array of studs.

23. The capacitive micromachined ultrasound transducer cell of claim 15, wherein sidewalls of the stud are vertical.

24. The capacitive micromachined ultrasound transducer cell of claim 15, wherein sidewalls of the stud are tapered.

25. The capacitive micromachined ultrasound transducer cell of claim 15, wherein sidewalls of the stud are rounded.

26. The capacitive micromachined ultrasound transducer cell of claim 9, wherein the at least one element formed in the cavity is a receding element.

27. The capacitive micromachined ultrasound transducer cell of claim 26, wherein the receding element is a well.

28. The capacitive micromachined ultrasound transducer cell of claim 27, wherein the well exhibits a circular shape.

29. The capacitive micromachined ultrasound transducer cell of claim 27, wherein the well exhibits a rectangular shape.

30. The capacitive micromachined ultrasound transducer cell of claim 27, wherein the well exhibits a hexagonal shape.

31. The capacitive micromachined ultrasound transducer cell of claim 27, wherein the well comprises a ring well.

32. The capacitive micromachined ultrasound transducer cell of claim 27, wherein the well comprises an array of wells.

33. The capacitive micromachined ultrasound transducer cell of claim 27, wherein sidewalls of the well are vertical.

34. The capacitive micromachined ultrasound transducer cell of claim 27, wherein sidewalls of the well are tapered.

35. The capacitive micromachined ultrasound transducer cell of claim 27, wherein sidewalls of the well are rounded.

36. A method for fabricating a capacitive micromachined ultrasound transducer cell, the method comprising:
forming a plurality of support posts on a lower electrode to define a cavity between the support posts;
forming at least one element in the cavity;
disposing a diaphragm on the plurality of support posts to form a gap between the lower electrode and the diaphragm; and
disposing an upper electrode on the diaphragm.

37. The method of claim 36, wherein forming the at least one element formed in the cavity comprises disposing one or more protruding elements formed in the cavity.

38. The method of claim 37, wherein the one or more protruding elements comprises a stud.

39. The method of claim 36, wherein forming at least one element formed in the cavity comprises disposing one or more receding elements formed in the cavity.

40. The method of claim 39, wherein the one or more receding elements is a well.

41. The method of claim 36, further comprising fabricating a bottom portion that comprises a lower electrode.

42. The method of claim 41, wherein fabricating the bottom portion comprises disposing a first oxide layer on a first side of a silicon layer.

43. The method of claim 41, further comprising disposing a second oxide layer on a second side of the silicon layer.

44. The method of claim 36, wherein forming a plurality of support posts comprises etching the second oxide layer to form the cavity.

45. The method of claim 44, further comprising disposing a third oxide layer on the silicon layer within the cavity.

46. The method of claim 36, further comprising fabricating a top portion that comprises an upper electrode.

47. The method of claim 46, wherein fabricating the top portion comprises disposing a first oxide box layer on a handle wafer.

48. The method of claim 47, further comprising disposing a conductive layer on a bottom side of the first oxide box layer, wherein the conductive layer comprises the diaphragm.

49. The method of claim 36, wherein the at least one element in the cavity comprises at least one of a stud and a well in the cavity.

50. The method of claim 36, wherein disposing a diaphragm on the plurality of support posts comprises disposing the top portion on the bottom portion via fusion bonding.

51. The method of claim 50, further comprising removing the handle wafer and the oxide box layer.

52. A capacitive micromachined ultrasound transducer cell structure, the structure comprising:
a first cell configured to operate in a receive mode, wherein the first cell comprises a lower electrode and an upper electrode;
a second cell configured to operate in a transmit mode disposed adjacent the first cell, wherein the second cell comprises a lower electrode and an upper electrode;
a plurality of support posts arranged to form cavities therebetween in each of the first cell and the second cell;
a plurality of diaphragms disposed on the support posts; and
at least one of a protruding element and a receding element formed in a cavity of one of the first cell and the second cell.

53. The capacitive micromachined ultrasound transducer cell of claim 52, further comprising at least one source of bias potential, wherein the at least one source of bias potential is configured to distend the diaphragms towards the lower electrodes.

54. The capacitive micromachined ultrasound transducer cell structure of claim 52, wherein the protruding element is a stud.

55. The capacitive micromachined ultrasound transducer cell of claim 54, wherein the stud exhibits a circular shape.

56. The capacitive micromachined ultrasound transducer cell of claim 54, wherein the stud exhibits a rectangular shape.

57. The capacitive micromachined ultrasound transducer cell of claim 54, wherein the stud exhibits a hexagonal shape.

58. The capacitive micromachined ultrasound transducer cell of claim 54, wherein the stud comprises a ring stud.

59. The capacitive micromachined ultrasound transducer cell of claim 54, wherein the stud comprises an array of studs.

60. The capacitive micromachined ultrasound transducer cell of claim 54, wherein sidewalls of the stud are vertical.

61. The capacitive micromachined ultrasound transducer cell of claim 54, wherein sidewalls of the stud are tapered.

62. The capacitive micromachined ultrasound transducer cell of claim 54, wherein sidewalls of the stud are rounded.

63. The capacitive micromachined ultrasound transducer cell structure of claim 52, wherein the receding element is a well.

64. The capacitive micromachined ultrasound transducer cell of claim 63, wherein the well exhibits a circular shape.

65. The capacitive micromachined ultrasound transducer cell of claim 63, wherein the well exhibits a rectangular shape.

66. The capacitive micromachined ultrasound transducer cell of claim 63, wherein the well exhibits a hexagonal shape.

67. The capacitive micromachined ultrasound transducer cell of claim 63, wherein the well comprises a ring shape.

68. The capacitive micromachined ultrasound transducer cell of claim 63, wherein the well comprises an array of studs.

69. The capacitive micromachined ultrasound transducer cell of claim 63, wherein sidewalls of the well are vertical.

70. The capacitive micromachined ultrasound transducer cell of claim 63, wherein sidewalls of the well are tapered.

71. The capacitive micromachined ultrasound transducer cell of claim 63, wherein sidewalls of the well are rounded.

72. The capacitive micromachined ultrasound transducer cell structure of claim 52, wherein the stud is disposed in the receive cell.

73. A capacitive micromachined ultrasound transducer cell structure of claim 52, wherein the well is etched in the transmit cell.

74. A method for fabricating a capacitive micromachined ultrasound transducer unit cell structure, the method comprising:
fabricating a first cell in the unit cell configured to operate in a receive mode, wherein the first cell comprises a lower electrode and an upper electrode; and
fabricating a second cell in the unit cell configured to operate in a transmit mode, wherein the second cell comprises a lower electrode and an upper electrode.

75. The method of claim 74, wherein the second cell is disposed adjacent to the first cell.

76. The method of claim 75, further comprising fabricating one of a protruding element and a receding element in one of the first cell and the second cell.

77. The method of claim 76, wherein the protruding element is a stud.

78. The method of claim 76, wherein the receding element is a well.

79. The method of claim 74, wherein fabricating at least one of the first and second cells comprises fabricating a bottom portion that comprises the lower electrode.

80. The method of claim 79, wherein fabricating the bottom portion comprises disposing a first oxide layer on a first side of a silicon layer.

81. The method of claim 80, further comprising disposing a second oxide layer on a second side of the silicon layer.

82. The method of claim 79, wherein fabricating the bottom portion comprises performing lithography and etching to define a cavity and the plurality of support posts.

83. The method of claim 79, further comprising disposing silicon adjacent to the plurality of support posts.

84. The method of claim 79, wherein fabricating the bottom portion comprises disposing a third oxide layer on the silicon layer within the cavity.

85. The method of claim 74, wherein fabricating at least one of the first and second cells comprises fabricating a top portion that comprises the upper electrode.

86. The method of claim 85, wherein fabricating the top portion comprises disposing a first oxide box layer on a handle wafer.

87. The method of claim 86, further comprising disposing a conductive layer on a bottom side of the first oxide box layer, wherein the conductive layer comprises the diaphragm.

88. The method of claim 74, wherein fabricating at least one of the first and second cells further comprises disposing the top portion on the bottom portion via fusion bonding.

89. The method of claim 88, wherein fabricating at least one of the first and second cells further comprises removing the handle layer via grinding and tetramethyl ammonium hydroxide, potassium hydroxide, or Ethylene Diamine Pyrocatechol etching.

90. The method of claim 74, wherein fabricating at least one of the first and second cells further comprises disposing the upper electrode on the diaphragm.

91. A system comprising:
a capacitive micromachined ultrasound transducer;
a resistor coupled to the capacitive micromachined ultrasound transducer;
a bias voltage bank coupled to the resistor;
a multiplexer coupled to the resistor;
a switch coupled to the multiplexer and configured to control modes of operation of the capacitive micromachined ultrasound transducer;
control circuitry coupled to the switch and configured to control operation of the bias voltage bank and the switch;
a pulser coupled to the switch and configured to generate alternating current excitation pulses; and
a low noise amplifier coupled to the switch and configured to enhance signals.

92. The system of claim 90, wherein the bias voltage bank comprises direct current to direct current converters.

93. The system of claim 90, wherein the bias voltage bank comprises application specific integrated circuit.

94. The system of claim 90, wherein the control circuitry comprises a programmable device.
